(19) United States
(12) Patent Application Publication
(10) Pub. No.: US 2009/0270977 A1
(43) Pub. Date: Oct. 29, 2009

(54) STENT FIXATION SYSTEM

(75) Inventors: Bettina Surber, Gachnang (CH); Daniel Wintsch, Zurich (CH); Tommy Conzelmann, Schaffhausen (CH); Nils Goetzen, Rostock (DE); Bodo Gerold, Zellingen (DE); Alexander Borck, Aurachtal (DE)

Correspondence Address: BRYAN CAVE POWELL GOLDSTEIN, ONE ATLANTIC CENTER FOURTEENTH FLOOR, 1201 WEST PEACHTREE STREET NW, ATLANTA, GA, 30309-3488, US

(73) Assignee: BIOTRONIK VI PATENT AG, Baar (CH)

(21) Appl. No.: 12/430,212

(22) Filed: Apr. 27, 2009

(30) Foreign Application Priority Data
Apr. 26, 2008 (DE) 10 2008 021 066.8

(51) Int. Cl. A61F 2/06

(52) U.S. Cl. 623/1.36

(57) ABSTRACT

A stent fixation system 3 for fixation of a stent, comprising two retaining areas 10 which are connected by a connecting area 12 to form a fixation tubing. The retaining areas 10 provide a retaining structure which is designed to hold at least one stent strut 2 of the stent in the undilated state, such that the stent struts 2 are no longer in one of the retaining areas 10, at least in the dilated state of the stent.

### PRIORITY CLAIM

This patent application claims priority to German Patent Application No. 10 2008 021 066.8, filed Apr. 26, 2009, the disclosure of which is incorporated herein by reference in its entirety.

### FIELD

The present disclosure relates to a fixation system for a stent.

### BACKGROUND

To an increasing extent, implants for reopening and supporting hollow organs such as blood vessels, urethras, bile ducts, uteruses and bronchi in the human body are used to an increasing extent in modern implantation medicine. For treatment of a stenosis in a blood vessel, balloon dilatation is first performed in a medical procedure and then a stent is inserted to prevent a renewed occlusion of the dilated vessel. In another procedure, the balloon dilatation is performed simultaneously with the placement of the stent. For this, an insertion system is mounted on the balloon catheter. With the help of various fixation methods, a stent is attached to the insertion system. After dilatation, the balloon is removed from the treated blood vessel, whereupon the stent remains at the site of dilatation.

To support the treatment, for some time now, stents have been coated with medications. These medications serve either to prevent or avoid inflammatory reactions of the vascular walls after the procedures (anti-inflammatory drugs) or excessive proliferation of smooth vascular muscle cells which can lead to a restenosis. Many substances also serve to accelerate the colonization of the stent with endothelial cells. This desired effect accelerates the growth of the stent into the vascular wall.

The stent must facilitate insertion by means of a catheter while fulfilling its function of vascular support with a large radial force at the site of the stenosis. For this, the stent in a compressed state is inserted as far as the site of treatment whereupon the stent is dilated. Dilatation of the stent may be accomplished with the help of a balloon on which the stent is mounted and which is then dilated by means of pressure, whereupon the stent widens uniformly. After releasing the pressure from the balloon, the stent remains in the dilated structure in the vessel. The alternative for balloon dilatation is the use of a stent made of a shape memory alloy such as Nitinol. The compressed stent is covered with an outer tubing. After introducing the stent to the site of the stenosis, the outer tubing is removed and the stent expands.

One technical challenge with the self-expandable systems is that compression of the stent can be prevented due to wall friction in retraction of the outer tubing. This compression occurs due to the friction between the outer tubing and the stent. A compressed stent tends to become entangled on the outer shaft, which thus increases the removal forces on the outer tubing.

A technical challenge of a balloon-expandable system is that the stent-balloon catheter system is coordinated in such a way that the stent does not slip on the insertion system during insertion into the vessel or the stent does not slip off completely but can be released easily without problems after being advanced to the treatment site.

Stent fixation systems are known from various publications, e.g., from U.S. Pat. No. 5,776,141, which describes retaining structures for stents and other tubular prostheses on balloon dilatation catheters. The stent here is also secured on the insertion system with the help of displaceable sheaths or ring-shaped displaceable retaining elements. Tearable strips pulled over the stent have also been described. The respective retaining element here covers the entire stent.

U.S. Pat. No. 4,950,227 describes a system in which a length of tubing which secures the stent on the balloon is pulled over the respective end of the stent. After advancing the stent to the treatment site, the lengths of tubing remain attached to the catheter and open out in a funnel shape. This solution may lead to the lengths of tubing either becoming entangled in the network structure of the stent, in the calcified vessels or in the guide catheter on retraction before or during the retraction of the catheter system from the vessels, thus making retraction much more risky. This pertains, in particular, to the distal length of tubing because the funnel opening here points in the direction of the retracting movement of the catheter system.

European Patent Application No. 1 785 107 describes a retaining system in which bands, open straps or stent struts running circumferentially in the form of a ring connect the catheter to the stent. The relatively long bands have the disadvantage that they become compressed on the inside of the curve and, therefore, protrude when the catheter system is retracted into curved vessels. As a result, the bands come in contact with the vascular walls and can, therefore, cause irritation to the tissue.

The approaches known in the past also have the disadvantage that so-called flaring occurs on both ends of the stent. Flaring refers to the protrusion of the ends of the stent in curved vascular paths. Due to its essentially elastic structure in the axial direction, the stent can follow almost any curvature in the vascular paths. However, the two stent ends tend toward elongation for purely mechanical reasons. This means that the stent ends tend to have less curvature. This produces contact with the vascular walls which can, in turn, prevent the advance of the system, on the one hand, while, on the other hand, causing inflammatory and, therefore, unwanted tissue reactions.

One feature of the present invention provides a stent fixation system which allows easy release of the stent and with which no retaining elements of any type remain in the vessel after the stent has been released in the vessel or become entangled in the vascular wall or the guide catheter on retraction.

### SUMMARY

The present disclosure describes several exemplary embodiments of the present invention.

One aspect of the present disclosure provides a stent fixation system, comprising a) a retaining structure defined by a first retaining area and a second retaining area b) a connection area connecting the first and second retaining areas so as to form a fixation tubing; and, c) at least one stent strut held, whereby the at least one stent strut can be maintained by the retaining structure when the stent is in an undilated state by the fixation tubing and whereby the at least one stent strut is substantially not in the first or second retaining areas when the stent is in a dilated state.

According to one exemplary embodiment, a stent fixation system 3 comprises a fixation tubing, which consists of two retaining areas 10 and a connecting area 12. The connecting area 12 connects the two retaining areas 10 to each other.

retaining areas 10 provide a retaining structure which is designed to hold at least one stent strut 2 of the stent in the undilated state such that the stent struts 2 are no longer in one of the retaining areas 10 in dilatation of the stent or at least in the dilated state of the stent. As soon as the stent is dilated and the distance between the individual stent struts 2 is increased, the stent struts 2 and retaining areas 10 are offset relative to one another so that the retaining area 10 no longer retains the stent struts 2.

The stent fixation system 3 of the present disclosure has the advantage that, mounted on an insertion system, the system brings the stent to the treatment site in the human or animal body in a manner that is safe and gentle for the vessels. This makes it possible to secure the stent on the stent fixation system 3 without the retaining structures interfering with the retraction of the insertion system or even remaining in the body after the stent has been released.

Due to the one-piece design of the fixation tubing, a small diameter of the stent fixation system 3 is ensured because no connecting seams (welds, solders or the like) are necessary. Retraction of the insertion system is thus facilitated by a small diameter of the stent fixation system 3. The insertion system may thus be removed from the body without causing excessive vascular irritation.

In the undilated state, the connecting area 12 of the fixation tubing is covered by the stent struts 2. The connecting area 12 may be foldable or may have slots which form a network-like structure after dilatation of the stent.

The fixation tubing is made of an elastic material, preferably a thermoplastic polyurethane (TPU). However, material selected from the group consisting of polyether block amides or rubber, in particular, silicone rubber, in combination with a medication would also be possible. The fixation tubing is slightly longer than the stent.

The stent fixation system 3 according to the present disclosure can be mounted on a dilatation catheter or the catheter shaft 1. The system can be attached either to the balloon, the neck of the balloon or the catheter shaft.

In addition, all or part of the stent fixation system 3 may be coated with an active pharmaceutical substance. It is advantageous, in particular, to coat the retaining structures of the two retaining areas 10 because they are the most likely to come in contact with the vascular walls 4 where they can release their pharmaceutical substance.

The active pharmaceutical substance is applied to the stent fixation system 3 by immersing, dipping, spraying, doctor blade application, diffusion, coating or pressing. It may either be applied alone or together with a vehicle or carrier.

By means of an adapted geometry with relatively short retaining areas 10, a higher lateral stability of the retaining area 10 is additionally achieved. This stability leads to controlled and/or reproducible release of the stent at the treatment site.

### BRIEF DESCRIPTION OF THE DRAWINGS

Various aspects of the present disclosure are described hereinbelow with reference to the accompanying figures.

FIG. 1 is a schematic view of one exemplary embodiment of a stent fixation system 3, which retains the stent 2 on the inner shaft 1 of a catheter, namely in the undilated state.

FIG. 2 is a schematic view of the stent fixation system of FIG. 1, no longer retaining the stent. The dilated stent 2 is in contact with the vascular wall 4.

FIG. 3 is a schematic view of the stent fixation system of FIG. 1 mounted on a dilatation balloon. The stent fixation system 3 retains the stent 2 on the dilatation balloon 5. The stent fixation system 3 and the dilatation balloon 5 are mounted on the inner shaft 1 of a catheter.

FIG. 4 is a schematic view of the stent fixation system of FIG. 1 with the dilatation balloon 5 in the dilated state.

FIG. 5 is a top schematic view of the stent fixation system of FIG. 1 with an undilated stent 2. The retaining areas 10 and the connecting area 12 are shown.

FIG. 6 is a top schematic view of the stent fixation system of FIG. 1 with a dilated stent 2. The retaining areas 10 and the connecting area 12 are also shown.

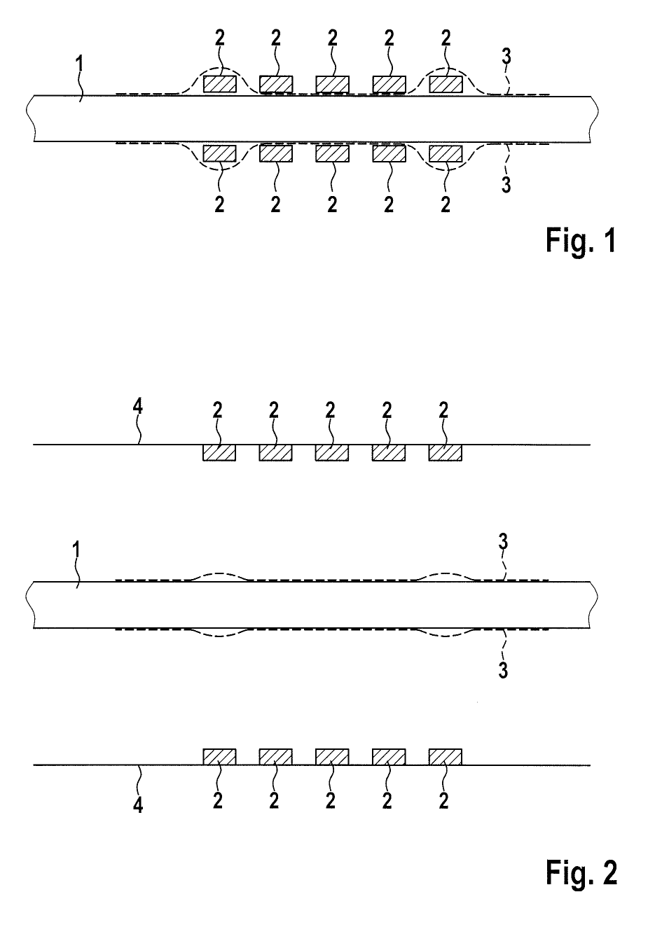

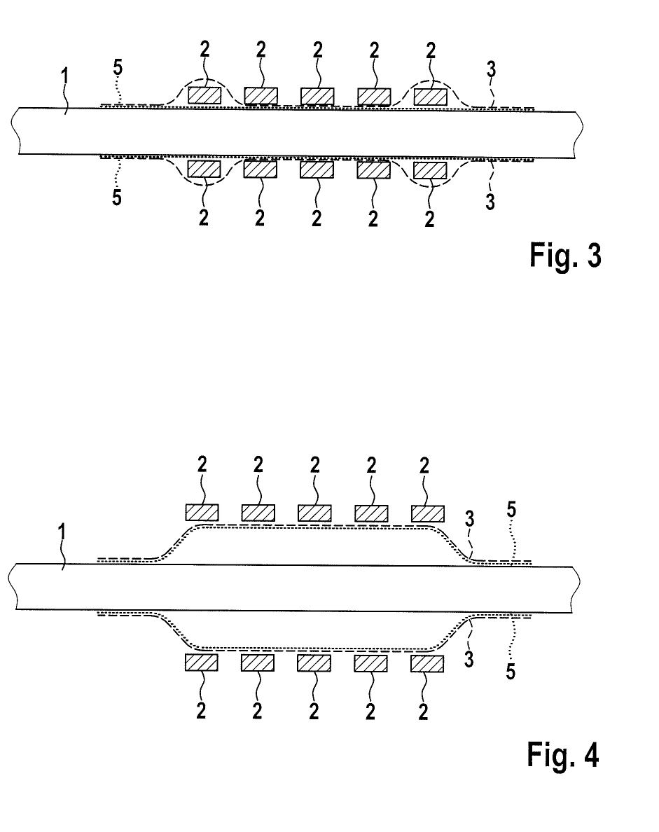

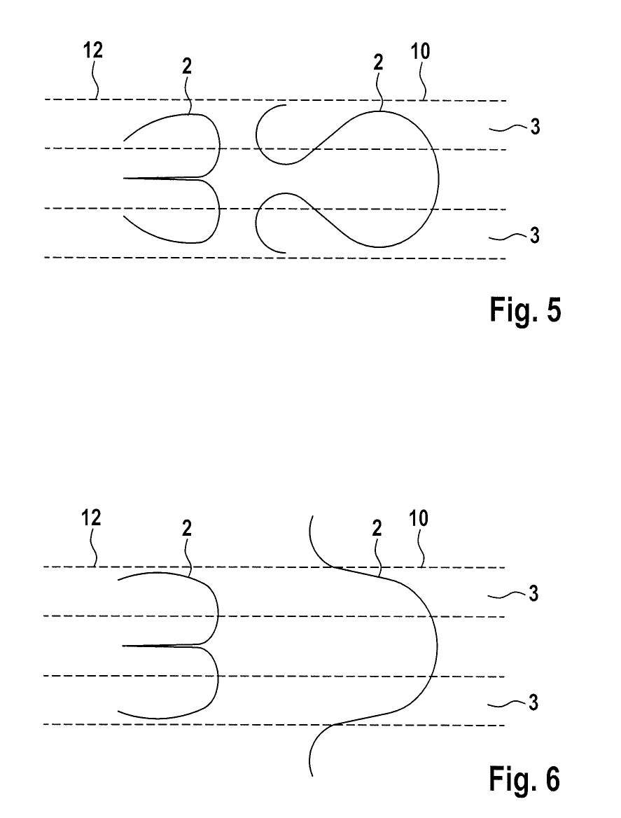

### DETAILED DESCRIPTION

One exemplary embodiment of the present disclosure is explained in greater detail hereinbelow. This preferred exemplary embodiment has a fixation tubing which is mounted on a catheter. This catheter has a dilatation balloon on its distal end for balloon dilatation of a stent.

The fixation tubing may be attached directly to the catheter shaft, the balloon neck or the balloon cylinder, for example. Depending on the combination of materials between the fixation tubing and the shaft, a suitable joining method (adhesive bonding, welding, riveting) is to be used. The stent is positioned on the fixation tubing such that the stent is secured by the retaining structure in the two retaining areas 10, and the connecting area 12 is beneath the stent.

The fixation tubing is made of an elastic material, preferably a thermoplastic polyurethane (TPU). However, material selected from the group consisting of polyether block amides or rubber, in particular, silicone rubber, would also be possible.

The two retaining areas 10 on the respective ends of the fixation tubing are secured by slots in the tubing in a defined form. The slots in the tubing create strip-type tubing parts which serve to secure one or more stent struts 2 by means of clamping forces in that these bands run over the stent struts 2.

The connecting area 12 of the tubing does not have any such slots and runs beneath the stent struts 2, which produces the fixation. This ensures that the stent will not slip on the catheter or even be lost on insertion into the human or animal body and on its passage through the vascular pathways.

When the catheter with the entire stent system has reached the site of treatment, the dilatation balloon 5 is dilated by a liquid introduced under pressure in a known manner. Since the fixation tubing together with the stent is securely connected to the dilatation balloon 5, it experiences the same radial force and is widened together with the dilatation balloon 5.

In the course of or at least at the end of dilatation, i.e., when the dilatation balloon 5 has reached its maximum diameter, the stent is widened so that the retaining structures of the retaining areas 10 no longer retain the stent but instead release it. The increasing distance between the stent struts 2 causes the strips to slip through in the retaining area 10 of the fixation tubing. The width of the bands is designed so that the stent is released as soon as possible, i.e., at the start of dilatation.

As an alternative, the connecting area 12 of the tubing may also have slots. A few slots at the respective ends of the fixation tubing serve to secure the stent. The slots in the connecting area 12 of the tubing are beneath the stent and thus do not have any retaining effect. The slots in the connecting area 12 may definitely have dimensions different (for example, longer) from the slots in the retaining area 10.

In dilatation of the balloon and thus the tubing, the result is a network-type structure of the connecting area 12. This has the advantage of increased elasticity of the fixation tubing. In release of pressure out of the dilatation balloon 5, the tubing retracts into a shape having a smaller radial diameter which thus facilitates retraction of the catheter system.

The slots may be created in the fixation tubing by various cutting methods, e.g., punching, laser cutting or water jet cutting. The manufacturing method is selected by those skilled in the art in accordance with the material used and the desired size of the slots. Alternatively, the slot structure can be implemented in production of the fixation tubing, e.g., through suitably designed injection molding.

The fixation tubing is attached to the catheter at the distal and proximal necks of the dilatation balloon 5 by adhesive bonding, welding or riveting. Depending on the combination of materials between the fixation tubing and the shaft, a suitable joining method (e.g., adhesive bonding, welding, and/or riveting) is to be used. When using a self-expanding stent, the fixation tubing is also attached to the inner shaft 1 of the catheter by adhesive bonding, welding or riveting, taking into account the compatibility of materials.

For implantation of a self-expandable stent, the fixation tubing is mounted directly on the inner shaft 1 of the catheter and fixedly attached to it by adhesive bonding, welding or riveting. The tubing, which is also slotted over the entire length or only in the retaining areas 10, is made of a material having a high strength but a low elastic expansibility, such as polyamide or a similar thermoplastic (HDPE).

The force acting in the direction of the vascular walls 4 comes only from the elastic restoring forces of the stent material. After retracting the exterior tubing over the stent, the latter expands and comes in close contact with the vascular walls 4. In doing so, the stent leaves the retaining bands of the retaining area 10 already at the start of dilatation. The fixation tubing here has the desired property of protecting the stent from compression during removal of the outside tubing when being pulled off.

Another exemplary embodiment relates to the design of the connecting area 12 of the fixation tubing. To achieve the required diameter following balloon dilatation, the connecting area 12 is arranged in folds beneath the stent like the dilatation balloon 5. In dilatation, the connecting area widens and the folds release the required amount of material. At the end of dilatation, the fixation tubing comes to lie directly above the dilatation balloon 5.

The stent fixation system 3 may be coated with at least one active pharmaceutical substance. The active pharmaceutical substance preferable is an active ingredient for treatment or prevention of in-stent restenosis, in particular, selected from the group consisting of: lipid regulators (fibrates), immunosuppressants, vasodilatators (sartans), calcium channel blockers, calcineurin inhibitors (tacrolimus), antiphlogistics (cortisone, diclofenac), anti-inflammatories (imidazoles), antiallergics, oligonucleotides (dODN), estrogens (genistein), endothelial cell-producing agents (fibrin), steroids, proteins/peptides, proliferation inhibitors (paclitaxel, sirolimus), analgesics, antirheumatics, angiogenesis inhibitors and cytostatics.

The active pharmaceutical substance is applied to the system, e.g., by immersion (immersion for a prolonged period of time and keeping the system immersed, optionally repeated), dipping (brief immersion and immediate removal, which may optionally also be repeated), spraying, doctor blade application, diffusion, coating or pressing.

The at least one active pharmaceutical substance is preferably applied together with a vehicle or carrier (e.g., a polymer or a solvent). In the case when a solvent is used, the solvent evaporates during and/or after application. When the solvent is expelled, the polymer carrier hardens at the same time. Depending on the active pharmaceutical substance, alcohols and other substances may be considered for use as the solvent, e.g., dimethyl sulfoxide (DMSO), acetone, ether (diethyl ether), methanol, isopropanol and esters.

It is advantageous if the active pharmaceutical substance with or without a carrier is applied only to the retaining areas 10 of the stent fixation system 3 because these are the areas that are most likely to come in contact with the vascular wall 4.

Since the stent fixation system 3 remains in the body for only a short period of time and, consequently, the substances on the retaining structures of the retaining areas 10 have only a short treatment time, it is advantageous if the system is provided with a coating, e.g., selected from the group consisting of sugars and fats, that can be washed off easily.

An especially preferred exemplary embodiment is to produce the stent fixation system 3 from silicone rubber (liquid silicone rubber, LSR). The active pharmaceutical substance is selected from the group consisting of paclitaxel, pimecrolimus and dexamethasone.

The two-component silicone material “silicone rubber” from Dow Corning (or Bayer), e.g., SILASTIC® 7-6840, is mixed 1:1 and reacted for 20 minutes at 100° C. The reaction may be carried out in the temperature range from 140° C. to 170° C. At these temperatures, the reaction time until curing amounts to one minute. Temperatures of 100° C. require longer curing times. Lower temperatures are preferred when using active pharmaceutical substances.

The active ingredient loading takes place by dissolving and/or preparing an extremely fine suspension of the active ingredient in one of the two components. Loads of up to 33% dexamethasone acetate are feasible in liquid silicone rubber.

Other materials for production of a stent fixation system 3 loaded with active ingredient include, for example: poly[ethylene-co-(vinyl acetate)]; acrylic polymers such as poly(methyl methacrylate) EUDRAGIT®, and PMMA; PEO/PEG; HPMA (N-(2-hydroxypropyl)methacrylamide); polyanhydrides (SEPTACIN®); polyesters such as PLLA, P₄HB, P₃HB, and PLGA; poly(orthoesters) and, poly(phosphoesters).

All patents, patent applications and publications referred to herein are incorporated by reference in their entirety.

What is claimed is:

1. A stent fixation system, comprising:
a) a retaining structure defined by a first retaining area and a second retaining area;
b) a connection area connecting the first and second retaining areas so as to form a fixation tubing; and,
c) at least one stent strut held,
whereby the at least one stent strut can be maintained by the retaining structure when the stent is in an undilated state by the fixation tubing and
whereby the at least one stent strut is substantially not in the first or second retaining areas when the stent is in a dilated state.

2. The stent fixation system of claim 1, wherein the fixation tubing is formed from a thermoplastic poly-urethane (TPU).

3. The stent fixation system of claim 1, wherein the connecting area has slots which form a network-like structure after dilatation of the stent.

4. The stent fixation system of claim 1, wherein the connecting area is foldable.

5. The stent fixation system of claim 1, wherein the stent fixation system is mountable either on a dilatation catheter or on an inner shaft of a catheter.

6. The stent fixation system of claim 1, wherein the total length of the fixation tubing is greater than the length of the stent.

7. The stent fixation system of claim 1, wherein the stent fixation system can be attached either to a balloon, to a balloon neck or to a catheter shaft.

8. The stent fixation system of claim 1, further comprising at least one active pharmaceutical substance applied either to the retaining structure or to the entire stent fixation system.

9. The stent fixation system of claim 8, wherein the at least one active pharmaceutical substance is applied either by immersion, dipping, spraying, doctor blade application, diffusion, coating or pressing.

10. The stent fixation system of claim 8, wherein the at least one active pharmaceutical substance is applied together with either a vehicle or carrier.

11. The stent fixation system of claim 8, wherein the at least one pharmaceutically active substance is a material selected from the group consisting of lipid regulators, immunosuppressants, vasodilatators, calcium channel blockers, calcineurin inhibitors), antiphlogistics, anti-inflammatories, antiallergics, oligonucleotides, estrogens, endothelial cell-producing agents, steroids, proteins, peptides, proliferation inhibitors, analgesics, antirheumatics, angiogenesis inhibitors and cytostatics.
